(19) United States
(12) Patent Application Publication
(10) Pub. No.: US 2012/0253066 A1
(43) Pub. Date: Oct. 4, 2012

(54) PROCESS FOR THE PREPARATION OF CILASTATIN SODIUM

(75) Inventors: Khadangale Bhausaheb Pandharinath, Aurangabad (IN); Kahandal Bhimraj Suryabhan, Aurangabad (IN); Suresh Pandian Pandi, Chennai (IN); Senthilkumar Udayampalayam Palanisamy, Chennai (IN); Sureshkumar Kanagaraj, Chennai (IN); Pandiarajan Sivasubramanian, Chennai (IN)

(73) Assignee: ORCHID CHEMICALS & PHARMACEUTICALS LIMITED, Chennai, Tamil Nadu (IN)

(21) Appl. No.: 13/512,243

(22) PCT Filed: Dec. 16, 2010
(86) PCT No.: PCT/IB2010/055870

§ 371 Date: May 25, 2012

(30) Foreign Application Priority Data
Jan. 1, 2010 (IN) 09/CHE/2010
Apr. 22, 2010 (IN) 1137/CHE/2010

(51) Int. Cl. C07C 61/35

(52) U.S. Cl. 562/506

(57) ABSTRACT

The present invention relates to an improved process for the preparation of Cilastatin Sodium of formula (I), having mesityl oxide content less than 2000 ppm and more than 1 ppm. (Formula I) (I)

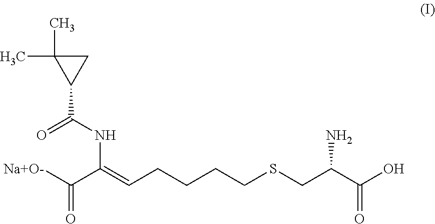

### FIELD OF THE INVENTION

The present invention relates to an improved process for the preparation of Cilastatin sodium of formula (I)

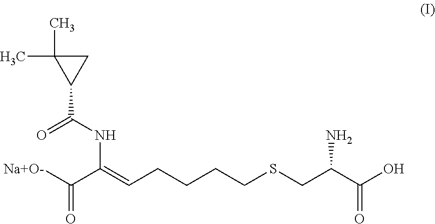

### DESCRIPTION OF THE PRIOR ART

Cilastatin sodium is the sodium salt of a derivatized heptenoic acid. Its chemical name is [R—[R*,S*-(Z)]]-7-[(2-amino-2-carboxyethyl)thio]-2-[[(2,2-dimethylcyclopropyl)carbonyl]amino]-2-heptenoic acid, monosodium salt. It is an off-white to yellowish-white, hygroscopic, amorphous compound. PRIMAXIN (Imipenem and Cilastatin) is a formulation of Imipenem (a thienamycin antibiotic) and Cilastatin sodium.

Imipenem with Cilastatin acts as an effective antibiotic for the treatment of infections of various body systems. PRIMAXIN is a potent broad-spectrum antibacterial agent for intramuscular administration. Imipenem can be further described as a semi-synthetic thienamycin that is administered intravenously or intramuscularly in combination with Cilastatin to reduce toxicity. Cilastatin, a renal dipeptidase inhibitor, inhibits the enzymatic breakdown of Imipenem and increases urinary excretion of the active drug.

Originally Cilastatin was disclosed in U.S. Pat. No. 5,147,868. This patent also discloses various processes for the preparation of Cilastatin, particularly example 19A of this patent disclose a process for the preparation of Cilastatin. According to this example the condensation of 7-chloro-2-oxoheptanoic acid ethyl ester (I) with (S)-2,2-dimethylcyclopropanecarboxamide (II) by means of p-toluene sulphonic acid in refluxing toluene gives (S)-7-chloro-2-(2,2-dimethylcyclopropanecarboxamido)-2-heptenoic acid ethyl ester (III), which is hydrolyzed in aq. NaOH to yield the corresponding carboxylic acid (IV). Finally, this compound is condensed with L-cysteine (V) by means of NaOH in water to afford the target Cilastatin, followed by isomerisation to at 3.0 pH. The process followed in this example is depicted as below:

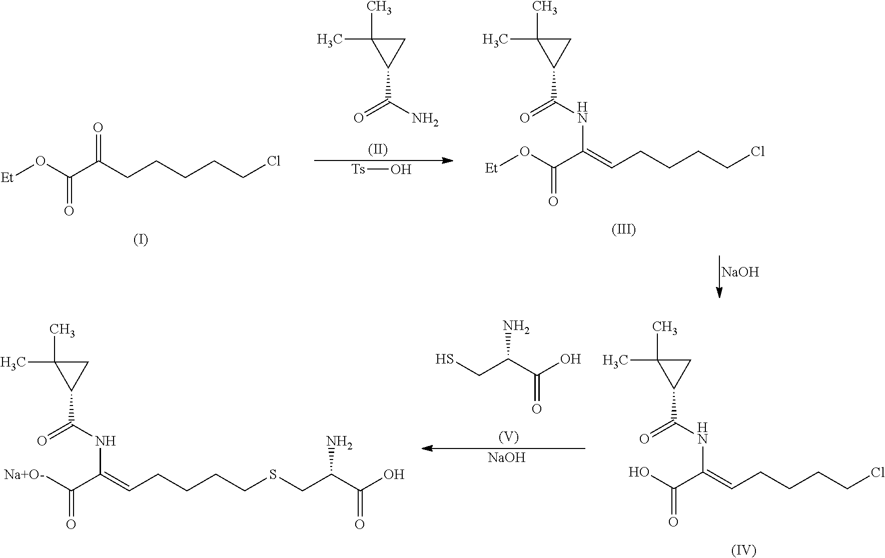

WO 03/018544 claims a process for the purification of Cilastatin, which comprises contacting a solution of crude Cilastatin with a non-ionic adsorbent resin and recovering pure Cilastatin from a solution thereof. This publication also claims a process for the isomerisation of Cilastatin by heating a solution of Cilastatin containing the corresponding E isomer at a pH of about 0.5 to 1.5. This invention not suitable for plant point of view as it involves column chromatography.

US 2004/0152780 claims a process for the preparation of pure Cilastatin sodium in an amorphous form which comprises recovering Cilastatin sodium from a solution thereof which contains an organic solvent, homogeneous mixture of organic solvents, or homogeneous mixture of organic solvents and water, by solvent precipitation. According to this patent the pure Cilastatin sodium in amorphous form was recovered from the solution of Cilastatin sodium in a solvent (where Cilastatin sodium was soluble) by adding an anti-solvent (where Cilastatin sodium was insoluble). The publication discloses the use of sodium hydroxide for the preparation of Cilastatin sodium from Cilastatin acid. The handling of said process is difficult in industrial scale as the quantity of sodium hydroxide to be used is directly related to assay value of Cilastatin acid. The presence of excess sodium hydroxide is precipitated along with cilastatin sodium and hence affects the pH of the reconstituted solution and presence of low quantity of sodium hydroxide affects the yield. Apart from this the use of sodium hydroxide as base and a ketonic solvent, particularly acetone as an anti-solvent yields Cilastatin sodium with higher amount of mesityl oxide impurity.

WO 2006/022511 claims a process for preparing Cilastatin sodium via Cilastatin amine salt, also the said patent claims Cilastatin ammonium salt. Also this patent utilizes the column chromatography for removing sodium chloride.

However taking the consideration the commercial importance of Cilastatin sodium and Imipenem, there remains a need of convenient process. In our continued research for developing a process for the preparation of Cilastatin sodium, we have identified a convenient process, which is commercially viable and also eliminates the foregoing problems associated with earlier processes.

### OBJECTIVE OF THE INVENTION

The primary objective of the present invention is to provide a simple, commercially viable process for the preparation of Cilastatin sodium of formula (I).

Another objective of the present invention is to provide a process for the preparation Cilastatin sodium with high purity and good yield which obviates the use of chromatography and provides direct isolation of Cilastatin sodium of formula (I).

Still another objective is to provide a process for the preparation of Cilastatin sodium of formula (I) having lower content of mesityl oxide impurity and pharmaceutically acceptable level of residual solvent.

Yet another objective of the present invention is to provide a process for the preparation of Cilastatin sodium of formula (I) having high purity, enhanced bulk density and improved flow properties.

### SUMMARY OF THE INVENTION

Accordingly, the present invention provides an improved process for preparation of Cilastatin sodium of formula (I)

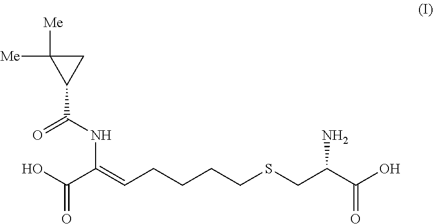

which comprises the steps of:
(a) reacting Cilastatin acid with sodium ion source in an organic solvent;
(b) optionally subjecting the reaction mass to carbon treatment;
(c) adding the reaction mass into a mixture of anti-solvent and water or vice-versa; and
(d) isolating the Cilastatin sodium.

### DETAILED DESCRIPTION OF THE INVENTION

In an embodiment of the present invention, the organic solvent employed in step (a) is selected from methanol, ethanol, propanol, n-butanol, denatured sprit, tetrahydrofuran, acetonitrile or mixtures thereof; preferably methanol.

In another embodiment of the present invention, the sodium ion source employed in step (a) selected from sodium alkoxide like sodium methoxide or sodium ethoxide, sodium acetate, sodium propionate, sodium citrate, sodium lactate, sodium 2-ethylhexanoate, sodium bicarbonate, sodium carbonate, sodium hydroxide or mixtures thereof. The sodium ion source is added directly to the reaction mass or used in the form of solution of sodium ion source in a solvent selected from the group consisting of water, methanol, ethanol, isopropyl alcohol, propanol, n-butanol, denaturated spirit, acetonitrile, tetrahydrofuran, acetone, methyl ethyl ketone or mixtures thereof.

In another embodiment of the present invention, the step (a) solution is optionally subjected to carbon treatment and micron filtration to obtain Cilastatin sodium as sterile product. Accordingly this present invention provides a process for the preparation of sterile Cilastatin sodium.

In yet another embodiment of the present invention, the anti-solvent employed in step (c) is selected from acetone, ethanol, 1-propanol, isopropyl alcohol, n-butanol, tertiary butyl alcohol, ethyl acetate, diisopropylether, denatured sprit, acetonitrile, tetrahydrofuran, methylene chloride or mixtures thereof; preferably acetone.

Though the above said solvent list used for precipitation of the Cilastatin sodium, the selection of anti-solvent has an effect in the yield, quality and flow properties of the final API. Considering all these factors, acetone is selected as an anti-solvent for crystallization in the conventional techniques; apart from the above acetone is effective and eco-friendly solvent. However it should be noted that use of acetone as an anti-solvent in the basic reaction medium leads to the self condensation and thereby results in high level of mesityl oxide impurity. It should be noted that as per pharmacopeia requirement mesityl oxide should not be present more than 4000 ppm in the final cilastatin sodium. Applicant surprisingly found that the selection of the anti-solvent containing water for (e.g. acetone containing water) for the crystallization of Cilastatin sodium leads to the minimization of associated impurities and provides Cilastatin sodium of formula (I) having lower content of mesityl oxide impurity. None of the prior art suggests this and constitutes advantage of the present invention. The addition of acetone containing water to reaction mass refers either acetone containing water is added to the Cilastatin sodium in a solvent or adding water to the solution of Cilastatin sodium followed by acetone. Accordingly the present invention provides Cilastatin sodium having of mesityl oxide less than 2000 ppm and greater than 1 ppm, preferably greater than 10 ppm.

In yet another embodiment of the present invention, the acetone:water ratio employed in step (c) is in the range of 99.9:0.1 to 90:10; preferably 99.9:0.1 to 95:5; more preferably 99.9:0.1 to 98:2.

In still another embodiment of the present invention, the Cilastatin sodium thus obtained is washed with mixture of two solvents selected from acetone, ethyl methyl ketone, ethanol, methanol, 1-propanol, isopropyl alcohol, n-butanol, ethyl acetate and the like; preferably mixture of ethanol and acetone.

Applicant found that use of single solvent for the washing of Cilastatin sodium leads to higher level of residual solvents, particularly acetone in the range of 4000 to 8000 ppm. Surprisingly applicant found that washing with mixture organic solvents results in lower content of acetone in the final product and also makes the final product with better flowability and good bulk density. Accordingly the present invention provides an improved process for reducing the acetone content in cilastatin sodium which comprises by treating the Cilastatin sodium having higher level of acetone content with mixture of two organic solvent selected from acetone, ethyl methyl ketone, ethanol, methanol, 1-propanol, isopropyl alcohol, n-butanol, ethyl acetate; preferably mixture of ethanol and acetone, followed by drying.

In one more another embodiment of the present invention, the cilastatin sodium obtained is optionally dried under nitrogen pressure or wet nitrogen or hot nitrogen pressure followed by vacuum drying at 40° C. to 80° C. for 6 to 15 hours. Accordingly, the present invention reduces the time of drying procedure.

In still another embodiment of the present invention, solution of Cilastatin sodium can be obtained by any conventional method or dissolving Cilastatin sodium in the solvent selected from water, methanol, ethanol, isopropyl alcohol and the like or mixtures thereof.

The starting material Cilastatin is prepared according to the procedure available in our patent application No. 1636/CHE/2005 dated Sep. 11, 2005 or by conventional methods or by reacting S-2,2-dimethylcylopropyl carboxamide with Ethyl-7-chloro-2-oxo-heptanoate to obtain ethyl-7-chloro-((S)-2,2-dimethylcyclopropanecarboxamido)-2-heptenoate. The resultant was subjected to isomerisation followed by hydrolysis and reacted with L-Cysteine hydrochloride monohydrate to yield Cilastatin acid.

Accordingly, (Z)-7-chloro-((S)-2,2-dimethylcyclopropanecarboxamido)-2-heptenoate is prepared by the isomerization of a mixture of ethyl (Z)-7-chloro-((S)-2,2-dimethylcyclopropanecarboxamido)-2-heptenoate and ethyl (E)-7-chloro-((S)-2,2-dimethylcyclopropanecarboxamido)-2-heptenoate; catalyzed with an acid selected from hydrochloric acid, sulphuric acid and the like. The ethyl (Z)-7-chloro-((S)-2,2-dimethylcyclopropanecarboxamido)-2-heptenoate obtained was alkali-hydrolyzed to obtain (Z)-7-chloro-((S)-2,2-dimethylcyclopropanecarboxamido)-2-heptenoic acid of high purity.

The present invention is exemplified by the following examples, which are provided for illustration only and should not be construed to limit the scope of the invention.

### EXAMPLE 1

### Preparation of Cilastatin Sodium

Cilastatin acid (40 g) was charged to a solution of sodium methoxide (20 g) in methanol (200 ml) and stirred at 25-30° C. The resultant solution was subjected to carbon treatment. The clear filtrate was filtered through 0.2 micron filter and slowly charged into acetone (1120 ml) containing water (5.6 ml). The material formed was filtered under nitrogen atmosphere. The wet cake was washed with 1:1 mixture of ethanol: acetone. The material was suck dried under nitrogen followed by drying under vacuum at 45-50° C.

### Yield: 39.7 g

Moisture content: 0.62% w/w
Content of mesityl oxide: 526 ppm (by Head Space GC).

### EXAMPLE 2

### Preparation of Cilastatin Sodium

Methanolic sodium methoxide solution (23 Kg) and Cilastatin acid (50 Kg) were taken in methanol. The pH of the reaction mixture was adjusted to 7 to 8 using sodium methoxide solution. The resultant solution was subjected to carbon treatment. The clear filtrate was filtered through 0.2 micron filter and slowly charged into acetone (1400 L) containing water (3.5 L). The material formed was stirred and filtered under nitrogen pressure. The wet cake was spray washed with acetone followed by slurry wash with 25:75 mixture of ethanol: acetone. The material was suck dried under nitrogen followed by drying under vacuum using hot water circulation at 65-70° C.

### Yield: 38.73 Kg

Purity: 99.34%, Content of mesityl oxide: 1440 ppm (by Head Space GC).

TABLE 1

| Experiment | Anti-solvent used | Content of mesityl oxide | |
|---|---|---|---|
| Reference example 2 | Acetone | Greater than 4000 ppm | |
| (Sodium hydroxide used | | | |
| as sodium ion donor) | | | |
| Reference example 2 | Acetone | 2874 | ppm |
| (Sodium methoxide used | | | |
| as sodium ion donor) | | | |
| Example 1 | acetone containing | 526 | ppm |
| | 0.5% water | | |
| Example 2 | acetone containing | 1404 | ppm |
| | 0.25% water | | |

From the above table-1, the use of acetone containing water brings down the mesityl oxide content to acceptable level.

TABLE 2

| Experiment | Solvent used for washing | Residual solvent data |
|---|---|---|
| Reference example 2 | Acetone | Acetone: 8932 ppm |
| Example 1 | Ethanol: acetone | Acetone: 1703 ppm |
| Example 2 | Ethanol: acetone | Acetone: 1838 ppm |

From the above table-2, use of a mixture of acetone and ethanol brings down the acetone content in the final API and reduce the drying time. Further it yields Cilastatin sodium with high purity, good yield and lower residual solvent content, which makes the invention more advantageous.

### REFERENCE EXAMPLE 1

### Preparation of Cilastatin Acid

To the solution of sodium hydroxide in Methanol, Chloro amido acid (50 gm) was charged followed by L-Cysteine hydrochloride (48.5 gm) and reaction mixture was refluxed for 3-4 hrs. After completion of reaction, pH was adjusted to 7-7.5 and filtered. The clear filtrate was distilled completely under vacuum. The residue obtained was dissolved in water and washed with MDC. To the aqueous layer n-butanol (7.0 vol.) was charged followed by the adjusting the pH to 3-3.5 at 25-30° C. The product was extracted into n-butanol. The n-butanol layer was washed with brine solution followed by water and distilled completely. The residue was dissolved in water. To the clear solution acetone (13 vol.) was added for precipitation and it was refluxed to 54-56° C. for 2.0 hrs. The obtained product was filtered and washed with acetone. The solid was again purified with aqueous acetone to yield pure Cilastatin acid in crystalline form.

### REFERENCE EXAMPLE 2

### Preparation of Cilastatin Sodium

A clear solution of Cilastatin sodium (obtained by treating Cilastatin acid in methanol with sodium ion source) was slowly added into acetone at 25 to 30° C. and stirred. The precipitated Cilastatin sodium was filtered and washed with acetone. The obtained Cilastatin sodium was optionally dried under nitrogen pressure, followed by drying under vacuum at 50 to 65° C.

Purity: 98.90%, Content of mesityl oxide: 2874 ppm (by Head Space GC), when sodium methoxide was used as a sodium ion source.
Content of mesityl oxide: greater than 4000 ppm (by Head Space GC), when sodium hydroxide was used as a as a sodium ion source.

1. Cilastatin sodium of formula (I) having mesityl oxide content less than 2000 ppm and more than 1 ppm,

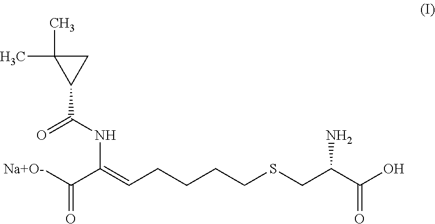

2. An improved process for the preparation of Cilastatin Sodium of formula (I), 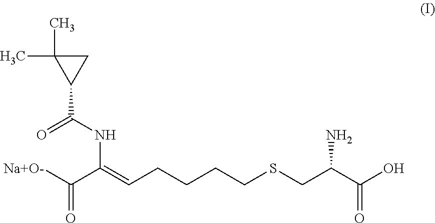
the process comprises the steps of:
reacting Cilastatin acid with sodium ion source in an organic solvent;
optionally subjecting the reaction mass to carbon treatment;
adding the reaction mass into a mixture of anti-solvent and water or vice-versa; and
isolating Cilastatin sodium.

3. The process as claimed in claim 2, wherein the sodium ion source used in step (a) of the reaction is selected from sodium methoxide, sodium ethoxide, sodium acetate, sodium propionate, sodium citrate, sodium lactate, sodium 2-ethylhexanoate, sodium bicarbonate, sodium carbonate, sodium hydroxide or mixtures thereof.

4. The process as claimed in claim 2, wherein the sodium ion source used in step (a) is sodium methoxide.

5. The process as claimed in claim 2, wherein the organic solvent employed in step (a) of the reaction is selected from methanol, ethanol, propanol, n-butanol, denatured sprit, tetrahydrofuran, acetonitrile or mixtures thereof.

6. The process as claimed in claim 2, wherein the organic solvent employed in step (a) of the reaction is methanol.

7. The process as claimed in claim 2, wherein the anti-solvent employed in step (c) is selected from acetone, ethanol, 1-propanol, isopropyl alcohol, n-butanol, tertiary butyl alcohol, ethyl acetate, diisopropylether, denatured sprit, acetonitrile, tetrahydrofuran, methylene chloride or mixtures thereof.

8. The process as claimed in claim 2, wherein the ratio of anti-solvent:water employed in step (c) is in the range of 99.9:0.1 to 90:10; preferably 99.9:0.1 to 98:2.

9. The process as claimed in claim 2, wherein the anti-solvent employed in step (c) is acetone.

10. The process as claimed in claim 2, further comprises washing the Cilastatin sodium with mixture of organic solvents selected from acetone, ethyl methyl ketone, ethanol, methanol, 1-propanol, isopropyl alcohol, n-butanol, ethyl acetate; preferably ethanol and acetone.

11. An improved process for the preparation of Cilastatin Sodium of formula (I) having mesityl oxide content less than 2000 ppm and more than 1 ppm, 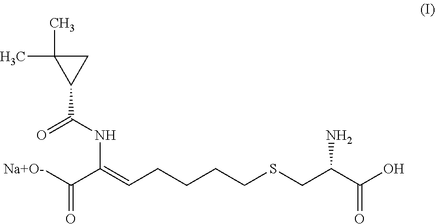
the process comprises the steps of:
reacting Cilastatin acid with sodium methoxide in an organic solvent;
optionally subjecting the reaction mass to carbon treatment;
adding the reaction mass into a mixture of acetone and water or vice-versa; and
isolating Cilastatin sodium.

12. (canceled)
